(19) United States
(12) Patent Application Publication
(10) Pub. No.: US 2015/0320842 A1
(43) Pub. Date: Nov. 12, 2015

(54) A COMPOSITION FOR PREVENTING OR TREATING AN RNA VIRAL INFECTION COMPRISING SAMHD1 OR A NUCLEIC ACID MOLECULE ENCODING THE SAMHD1

(71) Applicant: SNU R&DB FOUNDATION, Gwanak-gu, Seoul (KR)

(72) Inventors: Kwangseog AHN, Seoul (KR); Changhoon OH, Seoul (KR); Jongsu CHOI, Yongin-si, Gyeonggi-do (KR); Jeongmin RYOO, Seoul (KR)

(73) Assignee: SNU R&DB FOUNDATION, Seoul (KR)

(21) Appl. No.: 14/649,771

(22) PCT Filed: Jan. 7, 2013
(86) PCT No.: PCT/KR2013/000090

§ 371 Date: Jun. 4, 2015

(51) Int. Cl. A61K 38/46; C12N 15/11; A61K 31/7088

(52) U.S. Cl. CPC A61K 38/465; A61K 31/7088; C12N 15/11; C12Y 301/05

(57) ABSTRACT

The present disclosure relates to the ssRNA-specific ribonuclease activity of SAMHD1 (sterile alpha motif (SAM) domain and HD domain-containing protein 1) and its use. It is noteworthy that SAMHD1 has dual catalytic functions which are ribonuclease activity and dNTPase activity: however unlike the dNTPase activity, the ribonuclease activity of SAMHD1 did not require the cofactor dGTP. The present invention can be employed to degrade viral genomic ssRNA and to prevent or treat a ssRNA viral infection.

### BACKGROUND OF THE INVENTION

1. Field of the Invention

The present disclosure relates to the ssRNA-specific ribonuclease activity of SAMHD1 (sterile alpha motif (SAM) domain and HD domain-containing protein 1) and its use.

2. Description of the Related Art

Ribonucleases are enzymes that catalyze the degradation of RNA. A well studied ribonuclease is bovine pancreatic ribonuclease A (RNase A), the putative biological function of which is to break down the large amount of RNA that accumulates in the ruminant gut. Ribonucleases can be divided into endoribonucleases and exoribonucleases, and comprise several sub-classes within the EC 2.7 (for the phosphorolytic enzymes) and 3.1 (for the hydrolytic enzymes) classes of enzymes.

In the meantime, recent studies have reported that SAMHD1 possesses deoxynucleoside triphosphohydrolase (dNTPase) activity (Nature 480, 379-382, 2011). However, ribonuclease activity has not yet been described for this protein.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive researches on biological activity related with a virus replication of SAMHD1. As a result, we have found that the SAMHD1 had ability to degrade viral genomic single stranded RNA (ssRNA), and that this protein was able to inhibit viral replications.

Accordingly, it is an object of this invention to provide a pharmaceutical composition for preventing or treating a ssRNA viral infection.

It is an additional object of this invention to provide a method of preventing or treating a ssRNA viral infection in a subject in need thereof.

It is another object of this invention to provide a composition for degrading a viral genomic ssRNA.

It is still another object of this invention to provide a method of degrading a viral genomic ssRNA in a cell infected by a ssRNA virus.

It is still further another object of this invention to provide use of SAMHD1 or a nucleic acid molecule encoding the SAMHD1 for degrading a viral genomic ssRNA, or preventing or treating a ssRNA viral infection.

Other objects and advantages of the present invention will become apparent from the detailed description to follow taken in conjugation with the appended claims and drawings.

### BRIEF DESCRIPTION OF THE DRAWINGS

FIG. 1 shows that SAMHD1 is a single-stranded RNA-specific ribonuclease. [a] Purified GST-SAMHD1 (150 nM) was incubated for 30 min at 37° C. with various synthetic nucleic acid substrates (20-mers) that were 5′-end labeled with [γ-32P]ATP (indicated by an asterisk). The products were analyzed by electrophoresis and autoradiography. RNA and DNA are represented by red and blue lines, respectively. [b] GST-SAMHD1 was assayed for ribonuclease activity in time course experiments. [c] Ribonuclease activity of wild-type and putative catalytic mutants toward a 20-mer single-stranded RNA substrate labeled at the 5′-end.

FIG. 2 shows that SAMHD1 degrades RNA with a preference for a 3′-end containing a free hydroxyl group in a 3′ to 5′ direction. [a and b] GST-SAMHD1 was assayed for exoribonuclease activity in time course experiments. The RNA substrates were labeled with 32P at 3′-end. The 3′-end of the 30-mer substrate carried a hydroxyl group (a) or a phosphate group (b). The reaction products were separated by PAGE and visualized by autoradiography.

FIG. 3 shows that ribonuclease and dNTPase are dual and separable functions of SAMHD1. [a] A dGTPase activity assay was performed essentially as described previously (Nat Immunol 13, 223-228 (2012)). The reaction products generated upon incubation of GST-SAMHD1 proteins with dGTP were analyzed by polyethyleneimine cellulose thin layer chromatography. Unlabeled dGTP, dGDP, and dGMP were visualized using 254 nm ultraviolet and served as migration standards. PPP, triphosphate; PPP %, PPP/(dGTP+PPP) expressed as a percentage. [b] The ribonuclease activity assay was performed as described in FIG. 1s using ssRNA substrates that were 5′-end labeled with 32P.

FIG. 4 shows that the ribonuclease function of SAMHD1 is essential for HIV-1 restriction. [a] U937 cells stably expressing mock, wild-type or mutant SAMHD1 proteins with substitutions at the indicated positions were treated with PMA, followed by infection with HIV-1-GFP. After 48 hrs, these cells were subjected to whole-cell extraction before immunoblot analysis using the indicated antibodies. [b] The cells from (a) were harvested and then analyzed to determine the percentage of GFP-positive cells by flow cytometry. [b] The relative percentage of GFP-positive cells from (b) was calculated relative to the number of GFP-positive cells among the mock-transfected cells. Graphs show the mean±S.D. from three independent experiments. Two asterisks reflect a p value of <0.005. [d] The synthesis of viral cDNA intermediates was determined 24 hrs after infection by RT-PCR using R/U5 (early products, left panel) and U5/ψ primers (late products, right panel).

FIG. 5 shows that SAMHD1 directly degrades HIV-1 genomic RNA during infection. [a] The experimental design for time course analysis. SAMHD1-expressing U937 cells were infected with HIV-1-GFP and total cellular RNA was extracted at different time points post-infection. [b] Northern blot analysis of tRNA^{Lys3} levels using 18-mer nucleotide probe specific for tRNA^{Lys3} as a probe (upper panel). Equal loading of RNA samples is shown by ethidium bromide stain of gel before Northern blotting (lower panel). [c] HIV-1 genomic RNA content was quantified by qRT-PCR using HIV-1 gag-specific primer. The data were normalized to an internal control gene (β-actin). Similar results were obtained in three independent experiments. [d] U937 cells expressing vector alone, SAMHD1_{WT} or SAMHD1_{D207N} were infected for 90 min with HIV-1-GFP and crosslinked by formaldehyde. Cell lysates were immunoprecipitated with anti-HA antibody. Purified RNAs from the immunoprecipitates were quantified by qRT-PCR using HIV-1 gag-specific primer. [e] Role of SAMHD1 in HIV-1 restriction and AGS-related autoimmune diseases. Once HIV-1 genomic RNA is released from the core in the cytoplasm, SAMHD1 recognizes and directly degrades the HIV-1 genomic RNA. Effective clearance of HIV-1 by SAMHD1 may require the combined action of the ribonuclease and dNTPase activities (left). In human cells with missense mutations in SAMHD1, the RNA debris of endogenous retroviruses accumulates in the cytoplasm and is detected by sensors of antiviral innate immunity. The aberrant activation of innate immune response could cause pro-inflammatory diseases such as AGS (right).

### DETAILED DESCRIPTION OF THIS INVENTION

In one aspect of the present invention, there is provided a pharmaceutical composition for preventing or treating an RNA viral infection comprising a therapeutically effective amount of an RNA-degrading molecule as an active ingredient, wherein the RNA-degrading molecule is selected from SAMHD1 and a nucleic acid molecule encoding the SAMHD1.

In another aspect of this invention, there is provided a method of preventing or treating an RNA viral infection in a subject in need thereof, comprising administering to the subject a therapeutically effective amount of a pharmaceutical composition comprising an RNA-degrading molecule selected from SAMHD1 and a nucleic acid molecule encoding the SAMHD1.

In still another aspect of this invention, there is provided use of SAMHD1 or a nucleic acid molecule encoding the SAMHD1 as an RNA-degrading molecule for preventing or treating an RNA viral infection.

The present inventors have made intensive researches on biological activity related with a virus replication of SAMHD1. As a result, we have found that the SAMHD1 had ability to degrade viral genomic single stranded RNA (ssRNA), and that this protein was able to inhibit viral replications. The present invention is based on the new findings.

The term used herein “prevention” refers to the complete prevention of infection, the prevention of occurrence of symptoms in an infected subject, the prevention of recurrence of symptoms in an infected subject, or a decrease in severity or frequency of symptoms of viral infection, condition or disease in the subject.

The term “treatment” as used herein, refers to the partial or total elimination of symptoms or decrease in severity of symptoms of viral infection, condition or disease in the subject, or the elimination or decrease of viral presence in the subject.

The term used herein “subject” is intended to encompass human or non-human mammal or animal. Non-human mammals include livestock animals and companion animals, such as cattle, sheep, goats, equines, swine, dogs and cats.

As used herein, the term “therapeutically effective amount” means an amount of an RNA-degrading molecule which is sufficient, in the subject to which it is administered, to treat or prevent the symptoms, condition or disease related with viral infection.

The term “RNA-degrading molecule” as used herein refers to SAMHD1 and SAMHD1-encoding nucleic acids which are capable of degrading viral genomic ssRNAs.

The term used herein “RNA viral infection” refers to the entry of an ssRNA virus into a cell and the subsequent replication of the virus in the cell.

According to the present invention, the treatment strategies of the present invention are divided into two categories: (i) protein therapy or (ii) gene therapy.

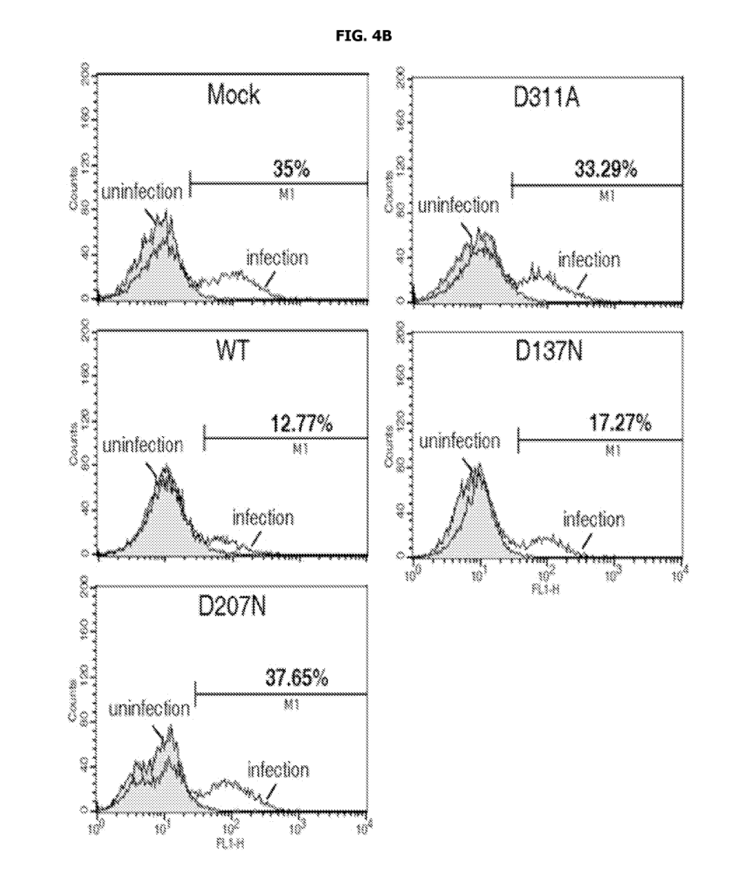

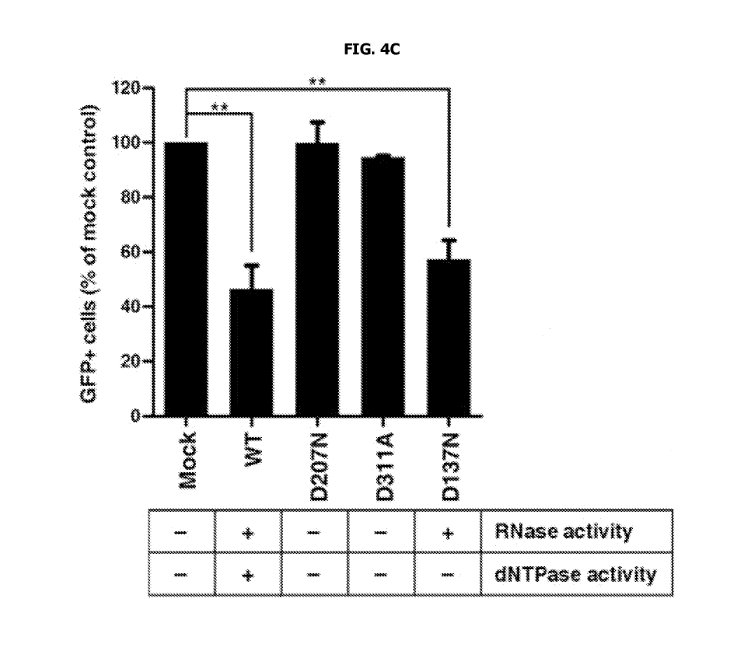

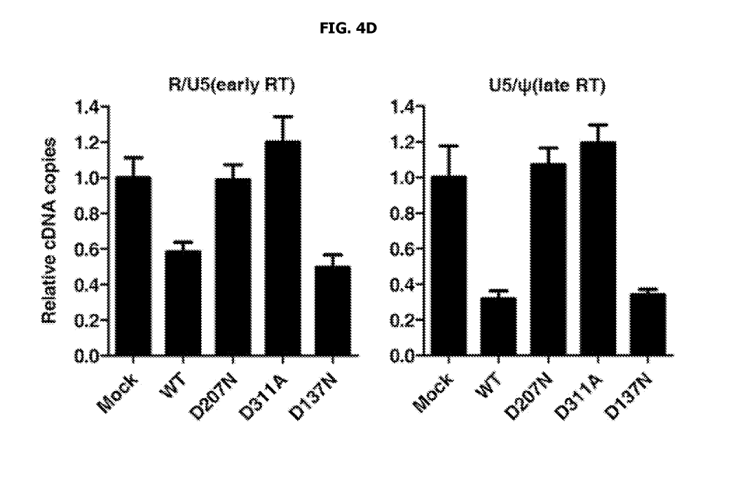

According to the protein therapy strategy of this invention, SAMHD1 (protein or polypeptide) is applied to the pharmaceutical composition as an active ingredient. As shown in FIG. 4b, SAMHD1 is non-toxic to the cells in which the SAMHD1 is introduced or overexpressed, indicating the SAMHD1 is ability to degrade specifically viral genomic ssRNA.

According to the gene therapy strategy of this invention, SAMHD1-encoding nucleic acid molecule is applied to the pharmaceutical composition as an active ingredient.

According to an embodiment, the nucleic acid molecule is a naked DNA or a component of gene delivery system. In addition, the pharmaceutical composition of this invention can be applied to a living body by various gene delivery methods known to gene therapy field. The term “gene delivery system” refers to any forms of carriers that harbor and transport exogenous nucleic acid molecules to a target cell. Specifically, the SAMHD1-encoding nucleotide sequence may be applied to a multitude of gene delivery systems useful in gene therapy, such as plasmid, adenovirus (Lockett L J, et al., Clin. Cancer Res. 3:2075-2080(1997)), adeno-associated virus (AAV, Lashford L S., et al., Gene Therapy Technologies, Applications and Regulations Ed. A. Meager, 1999), retrovirus (Gunzburg W H, et al., Retroviral vectors. Gene Therapy Technologies, Applications and Regulations Ed. A. Meager, 1999), lentivirus (Wang G. et al., J. Clin. Invest. 104(11):R55-62(1999)), herpes simplex virus (Chamber R., et al., Proc. Natl. Acad. Sci USA 92:1411-1415(1995)), vaccinia virus (Puhlmann M. et al., Human Gene Therapy 10:649-657(1999)), liposome (Methods in Molecular Biology, Vol 199, S. C. Basu and M. Basu (Eds.), Human Press 2002), neosome or nanoparticles.

(i) Adenovirus

Adenovirus has been usually employed as a gene delivery system because of its mid-sized genome, ease of manipulation, high titer, wide target-cell range, and high infectivity. The nucleotide sequence of interest to be delivered is inserted into either the deleted E1 region (E1A region and/or E1B region, preferably, EIB region) or the deleted E3 region, preferably, the deleted E1 region. Furthermore, the inserted sequences may be incorporated into the deleted E4 region.

According to an embodiment, the adenoviral gene delivery system of this invention comprises both “promoter-nucleotide sequence of interest-poly A sequence” and “promoter-SAMHD1 gene-poly A sequence”. The promoter-nucleotide sequence of interest-poly A sequence is present in either the deleted E1 region (E1A region and/or E1B region, preferably, EIB region) or the deleted E3 region, preferably, the deleted E1 region. The promoter-relaxin gene-poly A sequence is present in either the deleted E1 region (E1A region and/or E1B region, preferably, EIB region) or the deleted E3 region, preferably, the deleted E3 region. In addition, the adenoviral gene delivery system may comprise a bicistronic expression system in which the nucleotide sequence of interest and SAMHD1-encoding nucleotide sequence are linked each other by IRES (internal ribosome entry site) to form “promoter-nucleotide sequence of interest-poly A sequence-SAMHD1 gene-poly A sequence.

The foreign genes delivered by the present adenoviral gene delivery system are episomal, and therefore, have low genotoxicity to host cells. Therefore, gene therapy using the adenoviral gene delivery system of this invention may be considerably safe.

(ii) Retrovirus

Retroviruses capable of carrying relatively large exogenous genes have been used as viral gene delivery vectors in the senses that they integrate their genome into a host genome and have broad host spectrum.

In order to construct a retroviral vector, the SAMHD1-encoding nucleotide sequences and the nucleotide sequence of interest to be transferred are inserted into the viral genome in the place of certain viral sequences to produce a replication-defective virus. To produce virions, a packaging cell line containing the gag, pol and env genes but without the LTR (long terminal repeat) and IP components is constructed (Mann et al., Cell, 33:153-159(1983)).

A successful gene transfer using the second-generation retroviral vector has been reported. Kasahara et al. (Science, 266:1373-1376(1994)) prepared variants of moloney murine leukemia virus in which the EPO (erythropoietin) sequence is inserted in the place of the envelope region, consequently, producing chimeric proteins having novel binding properties. Likely, the present gene delivery system can be constructed in accordance with the construction strategies for the second-generation retroviral vector.

(iii) AAV Vector

Adeno-associated viruses are capable of infecting non-dividing cells and various types of cells, making them useful in constructing the gene delivery system of this invention. The detailed descriptions for use and preparation of AAV vector are found in U.S. Pat. Nos. 5,139,941 and 4,797,368.

Research results for AAV as gene delivery systems are disclosed in LaFace et al, Viology, 162:483486(1988), Zhou et al., Exp. Hematol. (NY), 21:928-933(1993), Walsh et al, J. Clin. Invest., 94:1440-1448(1994) and Flotte et al., Gene Therapy, 2:29-37(1995). Recently, an AAV vector has been approved for Phase I human trials for the treatment of cystic fibrosis.

Typically, a recombinant AAV virus is made by cotransfecting a plasmid containing the gene of interest (i.e. SAMHD1 gene and nucleotide sequence of interest to be delivered) flanked by the two AAV terminal repeats (McLaughlin et al., 1988; Samulski et al., 1989) and an expression plasmid containing the wild type AAV coding sequences without the terminal repeats (McCarty et al., J. Virol., 65:2936-2945(1991)).

(iv) Other Viral Vectors

Other viral vectors may be employed as a gene delivery system in the present invention. Vectors derived from viruses such as vaccinia virus (Puhlmann M. et al., Human Gene Therapy 10:649-657(1999); Ridgeway, “Mammalian expression vectors,” In: Vectors: A survey of molecular cloning vectors and their uses. Rodriguez and Denhardt, eds. Stoneham: Butterworth, 467-492(1988); Baichwal and Sugden, “Vectors for gene transfer derived from animal DNA viruses: Transient and stable expression of transferred genes,” In: Kucherlapati R, ed. Gene transfer. New York: Plenum Press, 117-148(1986) and Coupar et al., Gene, 68:1-10(1988)), lentivirus (Wang G. et al., J. Clin. Invest. 104(11):R55-62(1999)) and herpes simplex virus (Chamber R., et al., Proc. Natl. Acad. Sci USA 92:1411-1415(1995)) may be used in the present delivery systems for transferring both the SAMHD1 gene and nucleotide sequence of interest into cells.

(v) Liposome

Liposomes are formed spontaneously when phospholipids are suspended in an excess of aqueous medium. Liposome-mediated nucleic acid delivery has been very successful as described in Nicolau and Sene, Biochim. Biophys. Acta, 721:185-190(1982) and Nicolau et al., Methods Enzymol., 149:157-176(1987). Example of commercially accessible reagents for transfecting animal cells using liposomes includes Lipofectamine (Gibco BRL). Liposomes entrapping the SAMHD1 gene and nucleotide sequence of interest interact with cells by mechanism such as endocytosis, adsorption and fusion and then transfer the sequences into cells.

According to an embodiment, the SAMHD1-encoding nucleotide sequence is contained in a suitable expression construct. The term “expression construct” refers to any type of genetic construct comprising a nucleic acid coding for SAMHD1. According the expression construct, the SAMHD1-encoding nucleotide sequence is operatively linked to a promoter. The term “operatively linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence. According to the present invention, the promoter linked to the SAMHD1 gene is operable in, e.g. animal and mammalian cells, to control transcription of the SAMHD1 gene, including the promoters derived from the genome of mammalian cells or from mammalian viruses, for example, CMV (cytomegalovirus) promoter, the adenovirus late promoter, the vaccinia virus 7.5K promoter, SV40 promoter, HSV tk promoter, RSV promoter, EF1 alpha promoter, metallothionein promoter, beta-actin promoter, human IL-2 gene promoter, human IFN gene promoter, human IL-4 gene promoter, human lymphotoxin gene promoter and human GM-CSF gene promoter.

In an embodiment, the expression construct used in this invention comprises a polyadenylation sequence (e.g. bovine growth hormone terminator and SV40-derived polyadenylation sequence).

According to an embodiment, the expression construct for the SAMHD1-encoding nucleotide sequence has a structure of “promoter-SAMHD1-encoding nucleotide sequence-polyadenylation sequence.

According to an embodiment, the SAMHD1-encoding nucleic acid molecule is operatively linked to a promoter in an expression cassette. The term “expression cassette” refers to a nucleic acid molecule capable of directing expression of the SAMHD1-encoding nucleotide sequence in an appropriate host cell (e.g. human cells such as ssRNA virus-infected cells), comprising a promoter operatively linked to the nucleotide sequence of interest which is operatively linked to termination signals. It can be also include sequences required for proper translation of the nucleotide sequence.

According to an embodiment, SAMHD1 or SAMHD1-encoding nucleic acid molecule is derived from human. Their amino acid sequences and nucleotide sequences are disclosed in NCBI (National Center for Biotechnology Information). Examples of accession numbers of the SAMHD1 amino acid sequences are NM_{—}015474, EAW76090 and CAI42293. Examples of accession numbers of the SAMHD1 nucleotide sequences are NM_{—}015474. The human SAMHD1 has an amino acid sequence of SEQ ID NO:17.

According to the present invention, the RNA-degrading molecule is intended to include functional equivalents of SAMHD1 or SAMHD1-encoding nucleic acid molecule. As used herein, the term “functional equivalent” refers to amino acid sequence variants having amino acid substitutions, additions or deletions in some of the amino acid sequence of wild-type SAMHD1 while simultaneously having similar or improved biologically activity when compared to the SAMHD1, and refers to the amino acid sequence variant-encoding nucleotide sequences. The amino acid substitutions may be conservative substitutions. Examples of the conservative substitutions of naturally occurring amino acids include aliphatic amino acids (Gly, Ala, and Pro), hydrophobic amino acids (Ile, Leu, and Val), aromatic amino acids (Phe, Tyr, and Trp), acidic amino acids (Asp, and Glu), basic amino acids (His, Lys, Arg, Gln, and Asn), and sulfur-containing amino adds (Cys, and Met). The deletions of amino acids are located in a region which is not involved directly in the activity of SAMHD1.

According to the present invention, the amino acid sequences and nucleotide sequences of SAMHD1 available to the present invention are intended to include polynucleotide and polypeptide sequences having substantial identity to wild-type human SAMHD1 sequences. The term “substantial identity” as used herein means that the two nucleic acid or amino acid sequences, when optimally aligned, such as by the program BLAST, GAP or BESTFIT, or by visual inspection, share at least about 60%, 70%, 80%, 85%, 90% or 95% sequence identity or sequence similarity. Methods of alignment of sequences for comparison are well-known in the art. Various programs and alignment algorithms are described in: Smith and Waterman, Adv. Appl. Math. 2:482 (1981), Needleman and Wunsch, J. Mol. Bio. 48:443 (1970), Pearson and Lipman, Methods in Mol. Biol. 24: 307-31 (1988), Higgins and Sharp, Gene 73:237-44 (1988), Higgins and Sharp, CABIOS 5:151-3 (1989), Corpet et al., Nuc. Acids Res. 16:10881-90 (1988), Huang et al., Comp. Appl. BioSci. 8:155-65 (1992) and Pearson et al., Meth. Mol. Biol. 24:307-31 (1994) presents a detailed consideration of sequence alignment methods and homology calculations.

The NCBI Basic Local Alignment Search Tool (BLAST) (Altschul et al., J. Mol. Biol. 215:403-10 (1990)) is available from several sources, including the NCBI and on the internet, for use in connection with the sequence analysis programs blastp, blasm, blastx, tblastn and tblastx. It can be accessed at http://www.ncbi.nlm.nih.gov/BLAST/. A description of how to determine sequence identity using this program is available at http://www.ncbi.nlm.nih.gov/BLAST/blast_help.html.

According to the present invention, the RNA virus is a ssRNA virus selected from a group consisting of a retrovirus, a positive-sense ssRNA virus, a negative-sense ssRNA virus and an ambisense ssRNA virus. Animal RNA viruses are classified into two distinct groups depending on their genome and mode of replication: double-stranded RNA viruses and single stranded RNA viruses. The ssRNA viruses can be further classified according to the sense or polarity of their RNA into negative-sense and positive-sense, or ambisense RNA viruses. Positive-sense viral RNA is similar to mRNA and thus can be immediately translated by the host cell. Negative-sense viral RNA is complementary to mRNA and thus must be converted to positive-sense RNA by an RNA polymerase before translation. Ambisense RNA viruses resemble negative-sense RNA viruses, except they also translate genes from the positive strand. Retroviruses have also a ssRNA genome. The term “retrovirus” as used herein is defined as an RNA virus of the Retroviridae family.

Examples of retroviruses include, but are not limited to, members of the Lentivirus, Alpharetrovirus, Betaretrovirus, Gammaretrovirus, Deltaretrovirus, Spumavirus and Epsilonretrovirus.

According to an embodiment, the retroviruses include, but are not limited to, Human immunodeficiency virus such as HIV-1 and HIV-2, Simian immunodeficiency virus, Caprine arthritis encephalitis virus, Visna/maedi virus, Feline immunodeficiency virus, Puma lentivirus, Equine infectious anemia virus, Bovine immunodeficiency virus, Jembrana disease virus, Avian leukosis virus, Rous sarcoma virus, Jaagsiekte sheep retrovirus, Simian retrovirus, Mouse mammary tumor virus, Feline leukemia virus, Bovine leukemia virus, Walleye epidermal hyperplasia virus 1 and Walleye epidermal hyperplasia virus 2.

Examples of RNA viruses with positive-sense single stranded genome include, but are not limited to, members of the Arteriviridae, Coronaviridae, Roniviridae, Dicistroviridae, Iflaviridae, Marnaviridae, Picornaviridae, Secoviridae, Alphaflexiviridae, Betaflexiviridae, Gammaflexiviridae, Tymoviridae, Astrovirus, Barnaviridae, Bromoviridae, Caliciviridae, Closteroviridae, Flaviviridae, Leviviridae, Luteoviridae, Narnaviridae, Nodaviridae, Potyviridae, Togaviridae, Tombusviridae, Virgaviridae, Benyvirus, Cilevirus, Hepevirus, Idaeovirus, Ourmiavirus, Sobemovirus and Umbravirus.

According to an embodiment, the positive-sense ssRNA viruses include, but are not limited to, Equine arteritis virus, Lactate dehydrogenase elevating virus, Porcine reproductive and respiratory syndrome virus, Simian hemorrhagic fever virus, Coronavirus, Severe acute respiratory syndrome (SARS) coronavirus, Cripavirus, Deformed wing virus, Poliovirus, Human rhinoviruses, Hepatitis A virus, Avian nephritis virus, Norwalk virus, Yellow fever virus, West Nile virus, Hepatitis C virus, Dengue fever virus, Barley yellow dwarf virus, Rubella virus, Ross River virus, Sindbis virus, Chikungunya virus, Hepatitis E virus, Beet necrotic yellow vein virus, Beet soil-borne mosaic virus, Cocksfoot mottle virus, Lucerne transient streak virus and Rice yellow mottle virus.

Examples of RNA viruses with negative-sense single stranded genome include, but are not limited to, members of the Bornaviridae, Filoviridae, Paramyxoviridae, Rhabdoviridae, Arenaviridae, Bunyaviridae, Ophioviridae, Orthomyxoviridae, Deltavirus, Nyavirus and Tenuivirus.

According to an embodiment, the negative-sense ssRNA viruses include, but are not limited to, Borna disease virus, Ebola virus, Marburg virus, Measles virus, Mumps virus, Nipah virus, Hendra virus, Rabies virus, Lassa virus, Hantavirus, Citrus psorosis virus, Influenzavirus A, Influenzavirus B, Influenzavirus C and hepatitis D virus.

Examples of RNA viruses with ambisense genome include, but are not limited to, members of the bunyaviridae and arenaviridae.

According to an embodiment, the ambisense ssRNA viruses include, but are not limited to, Nairovirus, Orthobunyavirus, Phlebovirus, Tospovirus, Lymphocytic choriomeningitis (LCM) virus and Lassa fever virus.

According to an embodiment, the RNA-degrading molecule is exoribonuclease. In an embodiment, the RNA-degrading molecule is able to digest ssRNA processively in the 3′ to 5′ direction, i.e. 3′-5′ exoribonuclease. Accordingly, where the RNA-degrading molecule is introduced into cells infected by the ssRNA virus, it degrades the viral genomic ssRNA present in the cell, thereby inhibiting virus replication and eventually preventing or treating the viral infection.

According to an embodiment, the degradation of viral genomic ssRNA by the RNA-degrading molecule occurs in a cell cytoplasm.

According to an embodiment, the RNA viral ssRNA are degraded into 5 to 8-mer oligonucleotides by the RNA-degrading molecule.

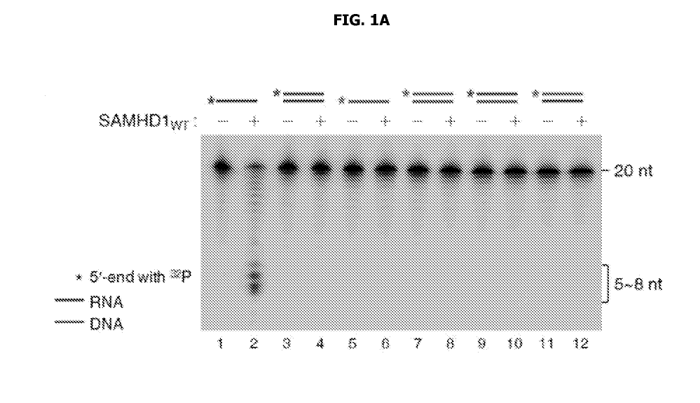

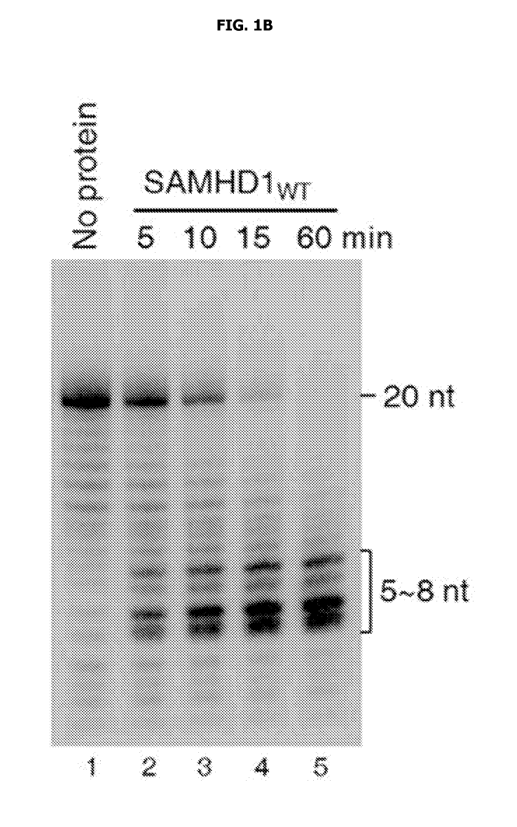

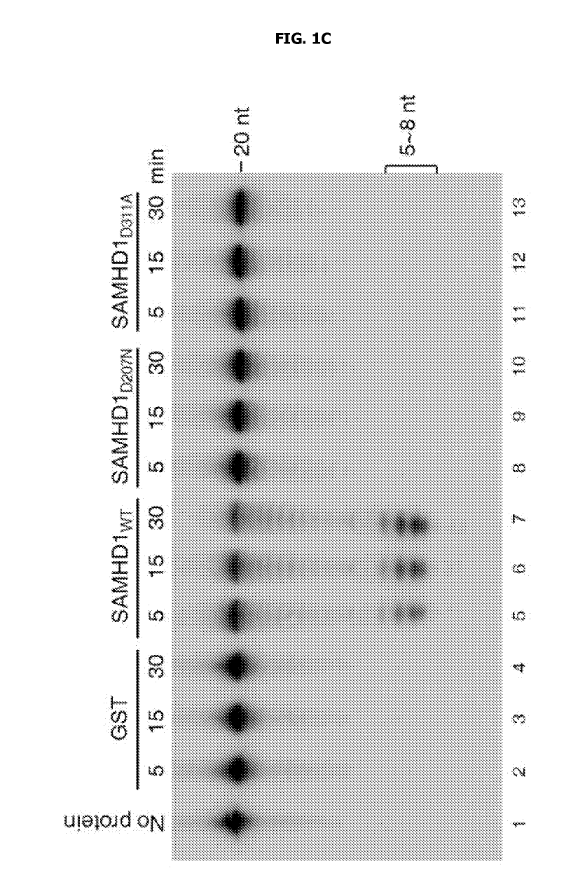

According to an embodiment, the RNA-degrading molecule is able to exhibit the ssRNA-specific ribonuclease activity without dGTP as a cofactor that is essential for the dNTPase activity. As shown in Examples below, we have first found that wild-type SAMHD1 has dual catalytic functions which are ribonuclease activity and dNTPase activity. It has been reported that the dNTPase function of SAMHD1 is allosterically activated by dGTP. However, as demonstrated in FIG. 1, unlike the dNTPase activity, the ribonuclease activity of SAMHD1 did not require the cofactor dGTP.

According to an embodiment, the SAMHD1 is lack of dNTPase activity.

According to an embodiment, the lack of dNTPase activity of SAMHD1 is achieved by conferring a mutation to the human SAMHD1.

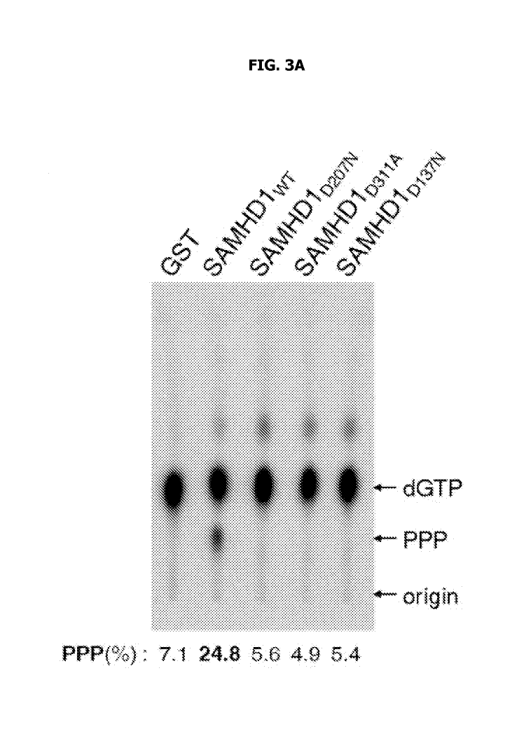

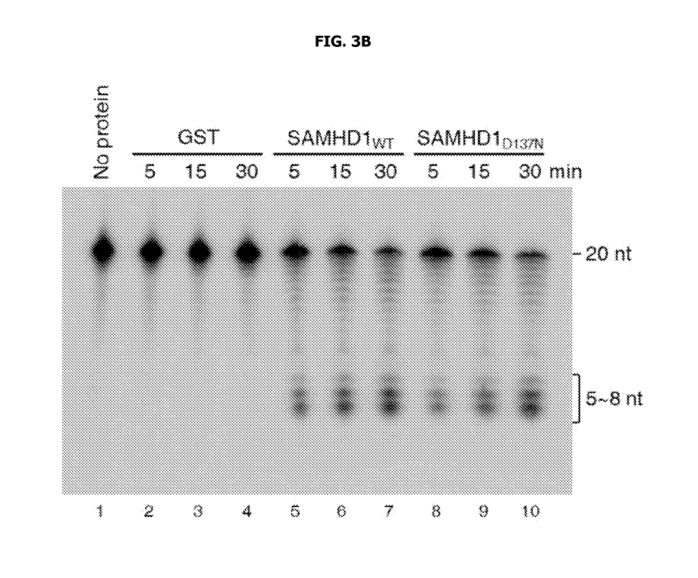

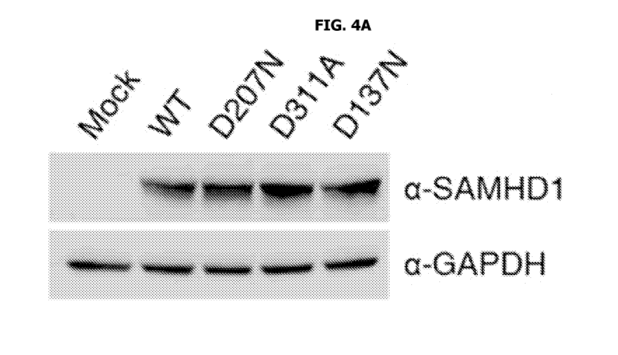

According to an embodiment, the SAMHD1 whose dNTPase activity is inactivated has the substitution mutation of Asn or Ala for Asp at position 137 of SEQ ID NO:17. As demonstrated in FIG. 3b, the mutation of amino acid 137 (D137N) in the allosteric site of SAMHD1 did not affect the ribonuclease activity, and the ribonuclease activity of this SAMHD1_{D137N} mutant was very similar to that of the wild-type SAMHD1.

According to the present invention, the pharmaceutical composition may contain pharmaceutically acceptable carriers. The pharmaceutically acceptable carrier may be conventional one for formulation, including lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils, but not limited to. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

The pharmaceutical composition according to the present invention may be administered via the oral or parenterally. When the pharmaceutical composition of the present invention is administered parenterally, it can be done by intravenous, subcutaneous, intramuscular, abdominal or transdermal administration.

A suitable dose of the pharmaceutical composition of the present invention may vary depending on pharmaceutical formulation methods, administration methods, the patient's age, body weight, sex, severity of diseases, diet, administration time, administration route, an excretion rate and sensitivity for a used pharmaceutical composition. the pharmaceutical composition of the present invention is administered with a daily dose of 0.0001-1,000 mg/kg (body weight).

According to the conventional techniques known to those skilled in the art, the pharmaceutical composition may be formulated with pharmaceutically acceptable carrier and/or vehicle as described above, finally providing several forms including a unit dose form and a multi-dose form. Formulation may be oil or aqueous media, resuspension or emulsion, extract, powder, granule, tablet and capsule and further comprise dispersant or stabilizer.

In yet another aspect of this invention, there is provided a composition for degrading a viral genomic ssRNA comprising an RNA-degrading molecule selected from SAMHD1 and a nucleic acid molecule encoding the SAMHD1.

In still yet another aspect of this invention, there is provided a method of degrading a viral genomic ssRNA in a cell infected by a ssRNA virus, comprising introducing to the cell an RNA-degrading molecule selected from SAMHD1 and a nucleic acid molecule encoding the SAMHD1, thereby degrading the viral genomic ssRNA.

In further another aspect of this invention, there is provided use of SAMHD1 or a nucleic acid molecule encoding the SAMHD1 as an RNA-degrading molecule for degrading a viral genomic ssRNA.

According to an embodiment, the virus is selected from a group consisting of a retrovirus, a positive-sense ssRNA virus, a negative-sense ssRNA virus and an ambisense ssRNA virus.

According to an embodiment, the nucleic acid molecule encoding SAMHD1, which is to be introduced into the cell, is a component of the expression construct mentioned above.

According to the present invention, any methods or techniques known in the art for delivering proteins or nucleic acids into cells (i.e. protein delivery system or gene delivery system) is utilized to introduce the RNA-degrading molecule into the cell infected by the ssRNA virus. Examples of the methods or techniques include, but are not limited to, microinjection method (Capecchi, M. R., Cell, 22:479(1980)), calcium phosphate precipitation method (Graham, F. L. et al., Virology, 52:456(1973)), electroporation (Neumann, E. et al., EMBO J., 1:841(1982)), liposome-mediated transformation method (Wong, T. K. et al., Gene, 10:87(1980)), DEAE-dextran treatment method (Gopal, Mol. Cell Biol., 5:1188-1190(1985)), and gene bombardment (Yang et al., Proc. Natl. Acad. Sci., 87:9568-9572(1990)).

The features and advantages of this invention will be summarized as follows:

(i) The present invention is directed to a single stranded RNA-degrading activity of SAMHD1 and its use.

(ii) It is noteworthy that SAMHD1 has dual catalytic functions which are ribonuclease activity and dNTPase activity: however unlike the dNTPase activity, the ribonuclease activity of SAMHD1 did not require the cofactor dGTP.

(iii) The present invention can be employed to degrade viral genomic ssRNA and to prevent or treat a ssRNA viral infection.

The present invention will now be described in further detail by examples. It would be obvious to those skilled in the art that these examples are intended to be more concretely illustrative and the scope of the present invention as set forth in the appended claims is not limited to or by the examples.

### Examples

### Experimental Methods

### Methods Summary

GST and GST-tagged proteins were expressed in E. coli Rosetta (ADE3) and purified using glutathione-agarose. After incubation of the recombinant proteins with synthetic 32P-labeled nucleic acid substrates, the reaction products were resolved using 15% polyacrylamide gels and were analyzed by autoradiography. For the dNTPase activity assay, recombinant proteins were incubated with [α-32P]dGTP and 200 μM cold dGTP, and the hydrolysis products were resolved by polyethyleneimine (PEI) cellulose thin-layer chromatography. Prior to HIV-1-GFP infection, U937 cells were differentiated overnight with 30 ng/ml PMA. Flow cytometry analysis of GFP expression was performed 2 days post-infection. The levels of HIV-1 genomic RNA were determined by quantitative RT-PCR.

### Plasmids

Human SAMHD1 was amplified by PCR from cDNA generated by the reverse transcription of RNA from HeLa cells, and the PCR product was inserted into pGEX-4T-1 vector and pMSCV-puro vector. The D207N, D311A and D137N mutants were generated using the nPfu-forte DNA Polymerase Kit (Enzynomics). HIV-1-GFP and HCMV-VSV-G were gift from Dan R. Littman (New York University School of Medicine).

### Protein Expression and Purification

Recombinant GST-fusion proteins were expressed in E. coli Rosetta (ADE3) (Novagen). Rosetta cells were grown in Terrific broth containing ampicillin (100 μg/ml) at 37° C. until an optical density (OD)₆₀₀ of 2.0 was reached, after which point the cells were quickly cooled on ice to 16° C. After induction with 0.1 mM isopropyl-β-D-thiogalacto-pyranoside (IPTG; Ducheba), the cells were allowed to grow for 16 hr at 16° C. The E. coli pellet containing the GST fusion protein was lysed with PBS, and the protein was purified using glutathione-Sepharose column chromatography, as previously described (Rohman, M. & Harrison-Lavoie, K. J. Protein Expr Purif 20, 45-47 (2000)).

### Substrate Preparation

Synthetic oligonucleotides were 5′-end labeled with ³²P using T4 polynucleotide kinase and [γ-³²P]ATP. Duplex substrates were prepared by incubating a 5′-end labeled oligonucleotide with the nonradioactive complementary oligonucleotide in a 1:1.2 molar ratio in the presence of 10 mM Tris-HCl (pH 8.0) and 20 mM KCl. The mixture was heated at 95° C. for 5 min and then allowed to cool slowly to room temperature. For 3′ labeling, an RNA oligonucleotide was incubated with [5′-³²P]pCp and T4 RNA ligase. The 3′ phosphate was removed by incubation with calf intestinal phosphatase. Following phenol/chloroform extraction, the RNA was precipitated. The sequences for all nucleic acid substrates are outlined in Table 1.

TABLE 1

| Oligomer | Sequence |
|---|---|
| 20-bp ssRNA | 5′-CUCCAUCACCCUCCAUCACC-3′ |
| | (SEQ ID NO: 1) |
| 30-bp ssRNA | 5′-ACCCACUGAAUCAGAUAAAGAGUUGUGUCA-3′ |
| | (SEQ ID NO: 2) |
| 20-bp dsRNA | 5′-CUCCAUCACCCUCCAUCACC-3′ |
| | (SEQ ID NO: 3) |
| | 3′-GAGGUAGUGGGAGGUAGUGG-5′ |
| | (SEQ ID NO: 4) |
| 20-bp ssDNA | 5′-CTCCATCACCCTCCATCACC-3′ |
| | (SEQ ID NO: 5) |
| 20-bp dsDNA | 5′-CTCCATCACCCTCCATCACC-3 |
| | (SEQ ID NO: 6) |
| | 3′-GAGGTAGTGGGAGGTAGTGG-5′ |
| | (SEQ ID NO: 7) |
| 20-bp RNA: | 5′-CUCCAUCACCCUCCAUCACC-3′ |
| DNA duplex | (SEQ ID NO: 8) |
| | 3′-GAGGTAGTGGGAGGTAGTGG-5′ |
| | (SEQ ID NO: 9) |

### In Vitro Nuclease Assay

Assays were carried out in 20 μl reaction mixtures containing PBS supplemented with 5 mM MgCl₂, 2 mM DTT, 10% glycerol, 0.01% NP-40, ³²P-labeled nucleic acid substrates, and purified recombinant proteins at 37° C. for the indicated time. RNA substrates labeled at the 3′-end with [5′-³²P]pCp were either untreated or treated with calf intestine phosphatase (CIP). Reactions were stopped with the addition of an equal volume of formamide loading buffer and then boiled. The products were separated in 15% polyacrylamide gels containing 8 M urea and buffered with 0.5× Tris-borate-EDTA (TBE) and then analyzed by autoradiography with a phosphorimager (BAS2500, Fujifilm).

dGTP-Triphosphohydrolase Assay

The enzymatic assay based on thin layer chromatography was performed as described previously (Lahouassa, H. et al. Nat Immunol 13, 223-228(2012)). In brief, the purified recombinant protein was incubated in 50 mM Tris-HCl (pH 8.0), 20 mM KCl, 5 mM MgCl₂, 0.1 μCi of the [α-³²P]dGTP, and 200 μM cold dGTP for 3 h at 37° C. The reactions were stopped by heat-inactivation at 70° C. for 10 minutes. The reaction mixtures were spotted together with standards of dGMP, dGDP, and dGTP onto polyethyleneimine (PEI)-cellulose plates (Sigma-Aldrich) and subsequently separated with a mobile phase of 1.2 M LiCl. Following separation, the α-³²P-labeled reaction products were visualized using a phosphorimager, and the migration indicators were detected by UV-C (254 nm).

### Cell Lines

U937 cells were cultured in RPMI 1640 supplemented with 10% fetal bovine serum (Hyclone), 2 mM GlutaMAX-I, 100 U/ml penicillin and 100 μg/ml streptomycin (Invitrogen). 293T cells and Phoenix Ampho cells were cultured in DMEM supplemented as described for RPMI 1640. Stable U937 cells expressing SAMHD1 proteins were obtained by retroviral infection. The pMSCV-puro construct was transfected into Phoenix Ampho cells by the calcium phosphate method. Two days later the retrovirus-containing supernatant was used to transduce U937 cells by spin infection in the presence of 8 μg/ml polybrene. Transduced cells were selected in 1 μg/ml puromycin.

### Virus Production and Infection

293T cells were transfected with 10 μg of HIV-1-GFP and 2 μg of HCMV-VSV-G using the calcium phosphate method. Virus-containing media was collected and filtered at 0.45 μm at 48 h after transfection. U937 cells (0.5×10⁶ cells/ml) were seeded in 12-well plates and differentiated for 20 h in PMA (30 ng/ml), and the viruses were added to target cells and incubated for 2 hrs. The cells were washed and then cultured for 48 h. Infected cells were analyzed by flow cytometry.

### Quantitative Real-Time PCR

RNA isolation from HIV-1-GFP-infected cells was performed at various time points according to the manufacturer's instructions (Invitrogen). A total of 1 μg RNA was reverse transcribed using random primers and real-time PCR was performed using the iCycler iQ real-time PCR detection system (BioRad) using the HIV-1-specific primers: R/U5 [forward (SEQ ID NO:10), reverse (SEQ ID NO:11)]; gag [forward (SEQ ID NO:12), reverse (SEQ ID NO:13)]; egfp [forward (SEQ ID NO:14), reverse (SEQ ID NO:15)].

### RNA Immunoprecipitation

HIV-1-GFP infected cells were crosslinked by 1% formaldehyde for 10 min at room temperature. Crosslinking reactions were stopped by the addition of glycine (1 M, pH 7.0) to a final concentration of 0.25 M followed by incubation at room temperature for 5 min. The cells were washed with ice-cold PBS and resuspended in RIPA buffer (50 mM Tris-HCl, pH 7.4/1% NP40/0.5% sodium deoxycholate/0.05% SDS/1 mM EDTA/150 mM NaCl) containing protease inhibitors and RNase inhibitor. The cell suspensions were sonicated and centrifuged at 13,000 rpm at 4° C. for 10 min to remove insoluble material. The remaining supernatants were precleared by incubation with protein G-agarose beads. The precleared supernatants were incubated with anti-HA antibody conjugated beads at 4° C. for 2 h. The beads were washed with RIPA buffer and resuspended with reversal buffer (50 mM Tris-HCl, pH 7.0/5 mM EDTA/10 mM DTT/1% SDS) followed by incubation at 70° C. for 45 min to reverse the crosslinks. The immunoprecipitated RNAs were isolated according to the manufacturer's protocol (Invitrogen).

Quantification of tRNA^{Lys3} by Northern Blot Analysis

Approximately 1 μg of total RNA was analyzed on 15% polyacrylamide gel containing 7 M urea after ethidium bromide staining. For Northern blot analysis, an oligomer probe specific for tRNA^{Lys3} (SEQ ID NO:16) was used, which is complementary to 18 nucleotide sequences at the 3′ end of the tRNA^{Lys3}. Briefly, the gel was electroblotted onto a positively charged nylon membrane at 20V for 2 hrs. Following cross-linking RNA to nylon membrane by using EDC (1-ethyl-3-(3-dimethylaminopropyl)carbodiimide), the membrane was baked in an oven at 80° C. overnight. Pre-hybridization and hybridization were subsequently carried out in the presence of 32P-5′-labelled nucleotide probes at 42° C. for 24 hrs. Following hybridization, the membrane was washed five times in 2×SSC (three times in 2×SSC containing 0.05% SDS and twice in 2×SSC containing 0.1% SDS) before autoradiography.

### Experimental Results

### SAMHD1 is a Single-Stranded RNA-Specific 3′-5′ Exoribonuclease

We explored the potential role of SAMHD1's nuclease activity in HIV-I restriction. The N-terminal GST-tagged full-length human SAMHD1 protein (GST-SAMHD1) was purified from E. coli and incubated with various types of 20-mer nucleic acid substrates. The substrates were labeled at 5′-end with ³²P and nuclease activity directed against nucleic acid substrates was assessed using gel electrophoresis. As shown in FIG. 1a, wild-type (WT) SAMHD1 specifically hydrolyzed single-stranded RNA (ssRNA), yielding a 5-8-mer as the major product. No nuclease activity was detected for double-stranded RNA (dsRNA), single-stranded DNA (ssDNA), double-stranded DNA (dsDNA), or RNA-DNA hybrid substrates (sequence information is provided in Table 1). This result indicates that SAMHD1 acts as an ssRNA-specific ribonuclease. To characterize the ribonuclease function of SAMHD1 more precisely, SAMHD1 was incubated for different periods of time with the same RNA labeled at its 5′-end with ³²P. This incubation resulted in the loss of the substrate and in the accumulation of 5- to 8-mer oligonucleotide products with a direct substrate/product relationship. No fragments of intermediate length were observed (FIG. 1b). This is a distinct feature of a processive rather than a distributive mode of hydrolysis. The HD domains possess putative nucleotidase and phophodiesterase activities, and the highly conserved His and Asp residues are critical for catalytic activity (Nature 480, 379-382 (2011)). We investigate whether these catalytic residues are required for ribonuclease activity of SAMHD1. An in vitro ribonuclease assay showed that the mutations at amino acids 207 and 311 in the catalytic site abolished the ribonuclease activity of the protein, indicating that these residues have the critical roles in the dNTPase and ribonuclease activities of SAMHD1

(FIG. 1c).

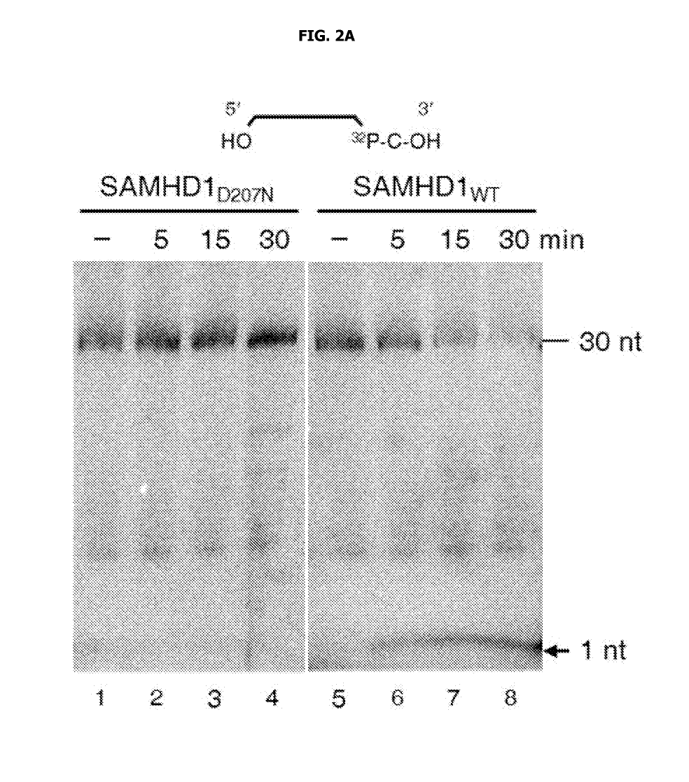

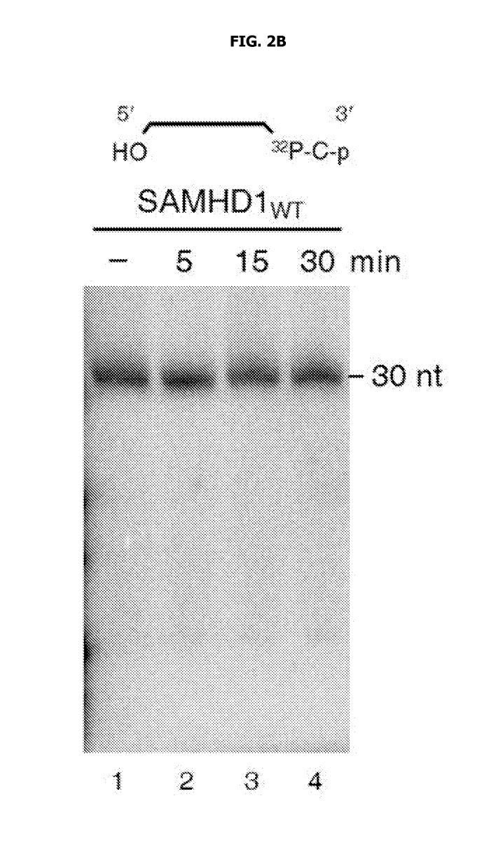

Next, to determine the direction of the degradation of ssRNA, SAMHD1 was incubated with another 30-mer ssRNA labeled with 32P at its 3′-end instead of its 5′-end. This incubation led to the loss of the signal, with no intermediate, and the signal of one nucleotide size was increased (FIG. 2a, right panel). As a control, the catalytically inactive SAMHD1_{D207N} mutant failed to degrade the substrate (FIG. 2a, left panel). By contrast, the substrate with a phosphate group at its 3′-end was not degraded (FIG. 2b). These results show that the SAMHD1-mediated degradation of ssRNA occurs in the 3′ to 5′ direction and displays a strong preference for a 3′-end containing a free hydroxyl group. The SAMHD1-mediated degradation of ssRNA is unlikely to be sequence specific based on the observation that both 20- and 30-mer “random” sequence ssRNAs were effectively targeted by SAMHD1. Taken together, our data demonstrate that SAMHD1 is a single-stranded RNA-specific exoribonuclease that processively digests RNA in the 3′ to 5′ direction.

Ribonuclease and dNTPase are Dual and Separate Functions of SAMHD1

The results of this and previous works (Nature 480, 379-382 (2011); J Biol Chem 286, 43596-43600 (2011)) suggest that ribonuclease activity and dNTPase activity are dual catalytic functions of SAMHD1. The dNTPase function of SAMHD1 is allosterically activated by dGTP. Mutations of Asp 137 and Arg 145 in the allosteric site or Asp 207 and Asp 311 in the catalytic site in the HD domain abolish dNTPase activity entirely 12. Because, unlike the dNTPase activity, the ribonuclease activity did not require the cofactor dGTP (FIG. 1), we proposed that these two catalytic functions of SAMHD1 might be distinguishable. To test this possibility, mutant with an Asn substitution in the allosteric site of SAMHD1 was constructed. GST-SAMHD1_{D137N} protein was produced in E. coli and purified by affinity chromatography. For unknown reasons, the production of another allosteric mutant substituted at Arg 145 failed despite numerous trials. The analysis of the dNTPase activity using dGTP as a substrate revealed that the mutations at amino acids 207, 311 and 137 abolished the dNTPase activity of SAMHD1 (FIG. 3a), consistent with the results of a previous study 12. Most interestingly, the mutation of amino acid 137 in the allosteric site did not abolish the ribonuclease activity (FIG. 3b). In contrast to the dNTPase activity, the ribonuclease activity of SAMHD1 appears not to involve the dGTP-mediated allosteric activation mechanism. These findings indicate that the ribonuclease activity of SAMHD1 is distinguishable from its dNTPase activity.

### The Ribonuclease Function of SAMHD1 is Critical for HIV-1 Restriction

The identification of a SAMHD1_{D137N} mutant that exhibits ribonuclease but no dNTPase activity enabled us to determine the discrete contribution of the ribonuclease activity of SAMHD1 to HIV-1 restriction. For this analysis, we stably expressed SAMHD1_{WT}, SAMHD1_{D207N}, SAMHD1_{D311A} or SAMHD1_{D137N} in U937 monocytic cells that do not express endogenous SAMHD1. After treatment of these cells with phorbol myristate acetate (PMA), they were infected with VSV-G pseudotyped HIV-1-GFP (hereafter HIV-1-GFP). The wild-type and mutant proteins were expressed at comparable levels in the HIV-1-GFP-infected cells (FIG. 4a). The SAMHD1_{WT} effectively inhibited HIV-I infection, whereas the catalytically inactive mutants (SAMHD1_{D207N} and SAMHD1_{D311A}) were essentially inactive for the restriction of HIV-1 infection (FIGS. 4b and 4c), and the results for the cells expressing these mutants were indistinguishable from the result for the mock-transfected control cells. Intriguingly, the SAMHD1_{D137N} allosteric mutant, which is devoid of dNTPase activity, still retained potent anti-HIV-1 restriction activity (79.7% of the SAMHD1_{WT} activity) (FIG. 4c). This result is in contrast to the complete loss of the restriction activity exhibited upon mutation of the two catalytic residues, D207 and D311. The SAMHD1_{D137N} displayed a modest but consistent 20.3% reduction relative to the SAMHD1_{WT} in its ability to restrict HIV-1-GFP (FIG. 4c), across a broad range of HIV-1-GFP doses. The lack of dNTPase activity may account for this 20.3% reduction in the anti-HIV-1 activity observed for the SAMHD1_{D137N}. Thus, we conclude, under our experimental conditions, that the ribonuclease function of SAMHD1 plays a major role in HIV-1 restriction. Quantification of viral cDNA intermediates by RT-PCR using the primers specific to the stages of HIV-1 reverse transcription showed that SAMHD1_{D137N} inhibited the synthesis of both the early and late viral cDNA products to a comparable extent as SAMHD1_{WT} (FIG. 4d), indicating that the ribonuclease function of SAMHD1 operates at the early step of HIV-1 reverse transcription.

SAMHD1 Associates with and Induces Destabilization of the HIV-1 Genomic RNA

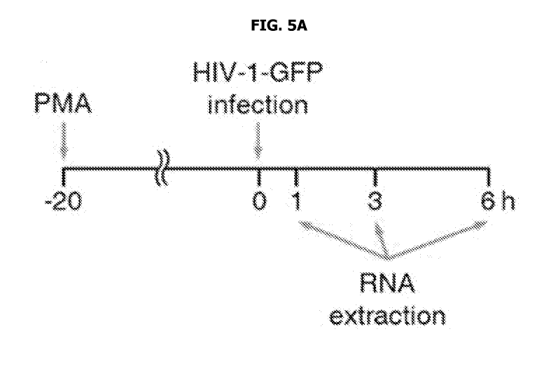

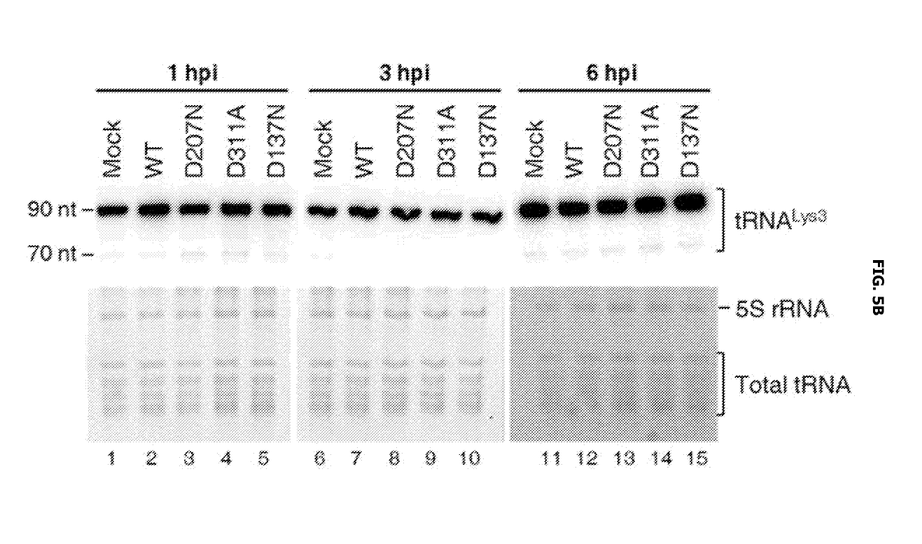

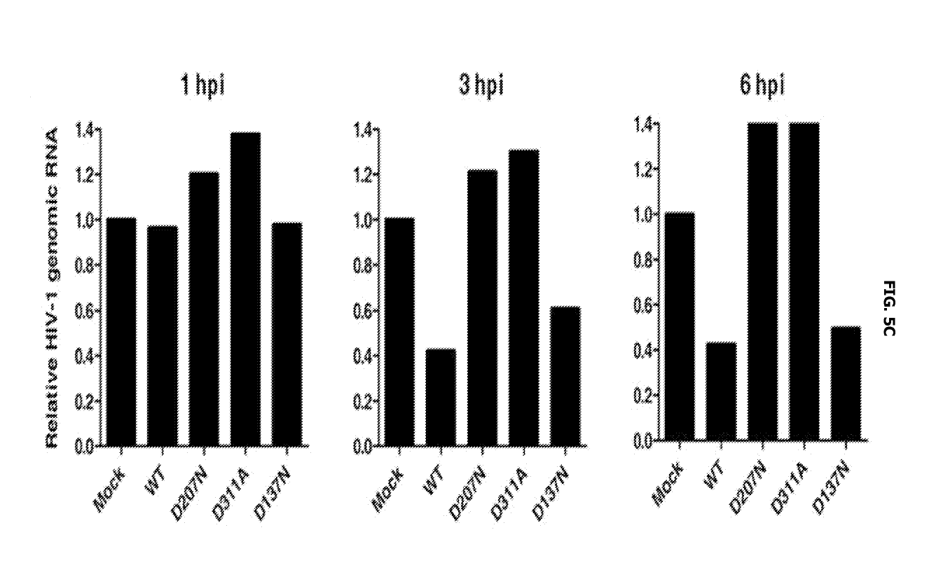

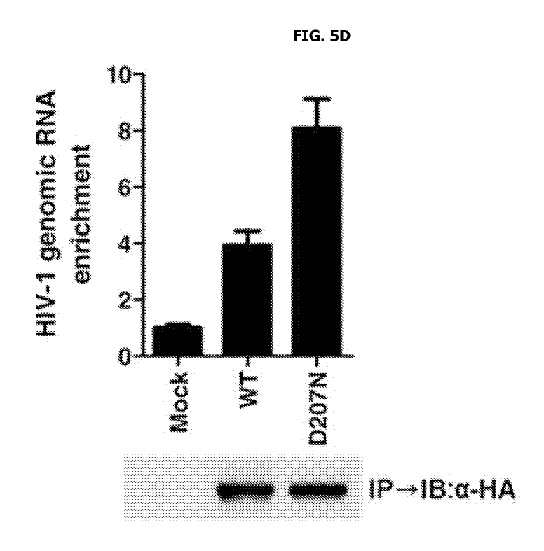

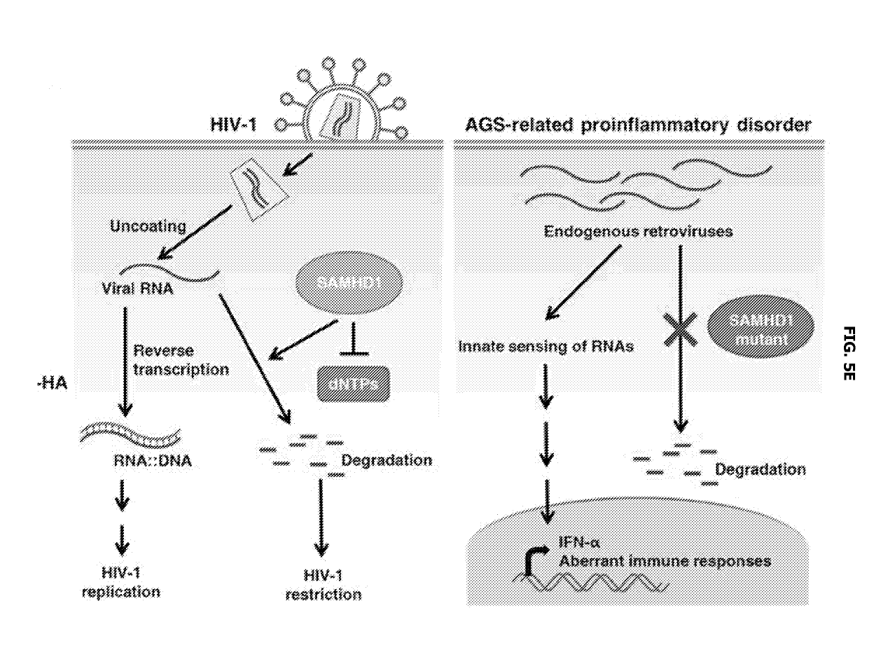

We investigated the mechanism by which SAMHD1 exerts its ribonuclease activity to inhibit HIV-1 replication. The experimental design is outlined in FIG. 5a. HIV-1 reverse transcription involves two RNA components, tRNA^{Lys3} as a primer and HIV-1 genomic RNA as a template for the synthesis of cDNA. Total cellular RNA was isolated from U937 cells infected with HIV-1-GFP at the early time points post-infection. Analysis by Northern blot revealed that SAMHD1 had no effect on the level of tRNA^{Lys3} (FIG. 5b, upper panel). In contrast, the quantification of viral RNA content by quantitative RT-PCR (qRT-PCR) using the HIV-1 gag-specific primer showed that at 3 and 6 hrs after infection, HIV-1-GFP genomic RNA was significantly reduced only in cells expressing SAMHD1_{WT} or SAMHD1_{D137N} (FIG. 5c). An analysis using the primers specific for different regions of HIV-1 genome showed essentially the same results. In particular, the SAMHD1_{D137N} allosteric mutant was able to reduce HIV-1 genomic RNA to levels almost comparable to the SAMHD1_{WT}, which correlates with the results obtained by FACS analysis (FIG. 4). Expression of SAMHD1_{D207N} or SAMHD1_{D311A} did not reduce HIV-1 genomic RNA compared with mock control. At 1 hr after infection, both SAMHD1- and SAMHD1_{D137N} had no impact on the viral RNA content (FIG. 5c), presumably due to the inaccessibility of SAMHD1 to the HIV-1 genomic RNA in the cores at this early time post-infection. To provide further evidence that SAMHD1 directly targets HIV-1 genomic RNA for degradation, we assessed whether SAMHD1 associated with HIV-1 genomic RNA during infection. We infected U937 cells expressing SAMHD1_{WT}-HA, SAMHD1_{D207N}-HA or vector alone with HIV-1-GFP for 90 min. The whole cell extracts were immunoprecipitated using anti-HA antibody. After RNA purification from the precipitates, qRT-PCR was performed to quantitate HIV-1 RNA. There was significant enrichment of HIV-1 RNA in cells expressing SAMHD1_{WT}-HA. The HIV-1 RNA enrichment was more pronounced for the SAMHD1_{D207N}-HA catalytic mutant with a 6- to 8-fold increase (FIG. 5d). Essentially the same results were obtained when quantification of the results was performed using the different primers (R/U5 and egfp), indicating that SAMHD1 associates with HIV-1 genomic RNA. Given the localization of substantial amounts of SAMHD1 to the cytoplasm, our data indicate that SAMHD1 binds and directly degrades HIV-1 genomic RNA in the cytoplasm where HIV-1 reverse transcription occurs.

### DISCUSSION

Our results demonstrate that SAMHD1 possesses a long-sought ribonuclease activity. Although the in vitro enzymatic analysis showed that SAMHD1 specifically degrades ssRNA, it remains elusive how SAMHD1 can recognize and degrade the secondary structured HIV-1 genomic RNA with distinct hairpin motifs in vivo. The putative helicase may be able to disrupt the secondary structure of the viral RNA for SAMHD1 ribonuclease function. Our findings, along with the previous findings for the dNTPase function of SAMHD1, indicate that SAMHD1 functions both as a ribonuclease and as a dNTPase. Interestingly, these two enzyme activities are separable as judged by the observations that the mutation of amino acid in the allosteric site abolishes the dNTPase but not the ribonuclease activity, and that the ribonuclease activity does not require a cofactor dGTP that is essential for the dNTPase activity. An intriguing question is why SAMHD1 evolves to have dual catalytic functions of SAMHD1 in a single enzyme towards restriction of HIV-1. HIV-1 can still replicate in non-cycling cells with low levels of dNTPs, such as macrophages and dendritic cells, albeit slowly and inefficiently (J Virol 68, 1258-1263 (1994); Proc Natl Acad Sci USA 90, 8925-8928 (1993)), most likely due to the ability of HIV-1's reverse transcriptase to bind dNTPs with high affinity (J Biol Chem 279, 51545-51553 (2004)). It was also known that reverse transcriptase of HIV-1 can use rNTP as substrate instead of dNTP in macrophage (J Biol Chem 285, 39380-39391). These studies suggest that it might not be sufficient to inhibit HIV-1 replication by the dNTPase activity alone of SAMHD1. Given the key roles of macrophages and dendritic cells as virus reservoirs in HIV-1 infection, the slow HIV-1 replication may be of physiological importance. Slowly replicating HIV-1 could sneak past immune surveillance and may be sufficient to allow long-term transmission to CD4+ T cells (Science 257, 383-387 (1992)). Effective clearance of HIV-1 by SAMHD1 may require the combined action of the ribonuclease and dNTPase activities.

Based on our data and the links between the accumulation of nucleic acids and the inappropriate triggering of innate immune responses, the natural function of SAMHD1 is likely to clean up dysfunctional cellular RNAs or RNA species derived from endogenous retroelement, thus preventing an unwanted inflammatory response. Human endogenous retroviruses make up nearly 8% of the human genome and have been implicated in some autoimmune diseases. In line with the specific recognition of HIV-1 RNA by SAMHD1 (FIG. 5) and the ability of SAMHD1 to restrict a wide range of retroviruses, the byproducts of human endogenous retroviruses are potentially major targets of SAMHD1. In addition, this mechanism may also explain the molecular basis of AGS and related lupus-like diseases in patients with loss-of-function mutations in SAMHD1. The failure to degrade endogenous RNA debris is likely a principle pathway causing the aberrant activation of innate immune responses in individuals with SAMHD1-mediated autoimmune diseases (FIG. 5e). The unique property of SAMHD1 that affects the balance between HIV-1 infection and innate immune response could be exploited to develop a new class of intervention for retroviruses and autoimmune diseases associated with nucleic acid metabolism.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A pharmaceutical composition for preventing or treating an RNA viral infection comprising a therapeutically effective amount of an RNA-degrading molecule as an active ingredient, wherein the RNA-degrading molecule is selected from SAMHD1 (SAM domain and HD domain-containing protein 1) and a nucleic acid molecule encoding the SAMHD1, and wherein the RNA virus is a single stranded RNA (ssRNA) virus selected from a group consisting of a retrovirus, a positive-sense ssRNA virus, a negative-sense ssRNA virus and an ambisense ssRNA virus.

2. The pharmaceutical composition of claim 1, wherein the retrovirus is selected from a group consisting of Lentivirus, Alpharetrovirus, Betaretrovirus, Gamma retrovirus, Deltaretrovirus, Spumavirus and Epsilonretrovirus.

3. The pharmaceutical composition of claim 2, wherein the retrovirus is selected from a group consisting of Human immunodeficiency virus, Simian immunodeficiency virus, Caprine arthritis encephalitis virus, Visna/maedi virus, Feline immunodeficiency virus, Puma lentivirus, Equine infectious anemia virus, Bovine immunodeficiency virus, Jembrana disease virus, Avian leukosis virus, Rous sarcoma virus, Jaagsiekte sheep retrovirus, Simian retrovirus, Mouse mammary tumor virus, Feline leukemia virus, Bovine leukemia virus, Walleye epidermal hyperplasia virus 1 and Walleye epidermal hyperplasia virus 2.

4. The pharmaceutical composition of claim 1, wherein the positive-sense ssRNA virus is selected from a group consisting of Arteriviridae, Coronaviridae, Roniviridae, Dicistroviridae, Iflaviridae, Marnaviridae, Picornaviridae, Secoviridae, Alphaflexiviridae, Betaflexiviridae, Gammaflexiviridae, Tymoviridae, Astrovirus, Barnaviridae, Bromoviridae, Caliciviridae, Closteroviridae, Flaviviridae, Leviviridae, Luteoviridae, Narnaviridae, Nodaviridae, Potyviridae, Togaviridae, Tombusviridae, Virgaviridae, Benyvirus, Cilevirus, Hepevirus, Idaeovirus, Ourmiavirus, Sobemovirus and Umbravirus.

5. The pharmaceutical composition of claim 4, wherein the positive-sense ssRNA virus is selected from a group consisting of Equine arteritis virus, Lactate dehydrogenase elevating virus, Porcine reproductive and respiratory syndrome virus, Simian hemorrhagic fever virus, Coronavirus, Severe acute respiratory syndrome (SARS) coronavirus, Cripavirus, Deformed wing virus, Poliovirus, Human rhinoviruses, Hepatitis A virus, Avian nephritis virus, Norwalk virus, Yellow fever virus, West Nile virus, Hepatitis C virus, Dengue fever virus, Barley yellow dwarf virus, Rubella virus, Ross River virus, Sindbis virus, Chikungunya virus, Hepatitis E virus, Beet necrotic yellow vein virus, Beet soil-borne mosaic virus, Cocksfoot mottle virus, Lucerne transient streak virus and Rice yellow mottle virus.

6. The pharmaceutical composition of claim 1, wherein the negative-sense ssRNA virus is selected from a group consisting of Bornaviridae, Filoviridae, Paramyxoviridae, Rhabdoviridae, Arenaviridae, Bunyaviridae, Ophioviridae, Orthomyxoviridae, Deltavirus, Nyavirus and Tenuivirus.

7. The pharmaceutical composition of claim 6, wherein the negative-sense ssRNA virus is selected from a group consisting of Borna disease virus, Ebola virus, Marburg virus, Measles virus, Mumps virus, Nipah virus, Hendra virus, Rabies virus, Lassa virus, Hantavirus, Citrus psorosis virus, Influenzavirus A, Influenzavirus B, Influenzavirus C and hepatitis D virus.

8. The pharmaceutical composition of claim 1, wherein the ambisense ssRNA virus is Bunyaviridae or Arenaviridae.

9. The pharmaceutical composition of claim 8, wherein the ambisense ssRNA virus is selected from a group consisting of Nairovirus, Orthobunyavirus, Phlebovirus, Tospovirus, Lymphocytic choriomeningitis (LCM) virus and Lassa fever virus.

10. The pharmaceutical composition of claim 1, wherein the SAMHD1 is lack of dNTPase activity.

11. The pharmaceutical composition of claim 10, wherein the SAMHD1 has the substitution mutation of Asn or Ala for Asp at position 137 of SEQ ID NO:17.

12. The pharmaceutical composition of claim 1, wherein the RNA-degrading molecule is able to degrade viral genomic ssRNA without dGTP as a cofactor.

13. The pharmaceutical composition of claim 1, wherein the SAMHD1-encoding nucleic acid molecule is operatively linked to a promoter in an expression cassette.

14. The pharmaceutical composition of claim 1, wherein the RNA-degrading molecule is 3′-5′ exoribonuclease.

15. The pharmaceutical composition of claim 1, wherein the RNA-degrading molecule degrades viral genomic ssRNA present in a cell cytoplasm.

16. The pharmaceutical composition of claim 1, wherein ssRNAs of the RNA virus are degraded into 5 to 8-mer oligonucleotides by the RNA-degrading molecule.

17. A composition for degrading a viral genomic ssRNA comprising an RNA-degrading molecule selected from SAMHD1 and a nucleic acid molecule encoding the SAMHD1, wherein the virus is selected from a group consisting of a retrovirus, a positive-sense ssRNA virus, a negative-sense ssRNA virus and an ambisense ssRNA virus.

18. A method of preventing or treating an RNA viral infection in a subject in need thereof, comprising administering to the subject a therapeutically effective amount of a pharmaceutical composition comprising an RNA-degrading molecule selected from SAMHD1 and a nucleic acid molecule encoding the SAMHD1, wherein the RNA virus is a ssRNA virus selected from a group consisting of a retrovirus, a positive-sense ssRNA virus, a negative-sense ssRNA virus and an ambisense ssRNA virus.

19. A method of degrading a viral genomic ssRNA in a cell infected by a ssRNA virus, comprising introducing to the cell an RNA-degrading molecule selected from SAMHD1 and a nucleic acid molecule encoding the SAMHD1, thereby degrading the viral genomic ssRNA, wherein the virus is selected from a group consisting of a retrovirus, a positive-sense ssRNA virus, a negative-sense ssRNA virus and an ambisense ssRNA virus.

20. Use of SAMHD1 or a nucleic acid molecule encoding the SAMHD1 as an RNA-degrading molecule for preventing or treating an RNA viral infection, wherein the RNA virus is a ssRNA virus selected from a group consisting of a retrovirus, a positive-sense ssRNA virus, a negative-sense ssRNA virus and an ambisense ssRNA virus.

21. Use of SAMHD1 or a nucleic acid molecule encoding the SAMHD1 as an RNA-degrading molecule for degrading a viral genomic ssRNA, wherein the virus is selected from a group consisting of a retrovirus, a positive-sense ssRNA virus, a negative-sense ssRNA virus and an ambisense ssRNA virus.


SEQUENCE LISTING (17 sequences)

SEQ ID NO: 1 - RNA, 20 nt, Artificial Sequence
  20-bp ssRNA
cuccaucacccuccaucacc

SEQ ID NO: 2 - RNA, 30 nt, Artificial Sequence
  30-bp ssRNA
acccacugaaucagauaaagaguuguguca

SEQ ID NO: 3 - RNA, 20 nt, Artificial Sequence
  20-bp dsRNA 5′-3′
cuccaucacccuccaucacc

SEQ ID NO: 4 - RNA, 20 nt, Artificial Sequence
  20-bp dsRNA 3′-5′
gagguagugggagguagugg

SEQ ID NO: 5 - DNA, 20 bp, Artificial Sequence
  20-bp ssDNA
ctccatcaccctccatcacc

SEQ ID NO: 6 - DNA, 20 bp, Artificial Sequence
  20-bp dsDNA 5′-3′
ctccatcaccctccatcacc

SEQ ID NO: 7 - DNA, 20 bp, Artificial Sequence
  20-bp dsDNA 3′-5′
gaggtagtgggaggtagtgg

SEQ ID NO: 8 - RNA, 20 nt, Artificial Sequence
  20-bp RNADNA duplex 5′-3′
cuccaucacccuccaucacc

SEQ ID NO: 9 - DNA, 20 bp, Artificial Sequence
  20-bp RNADNA duplex 3′-5′
gaggtagtgggaggtagtgg

SEQ ID NO: 10 - DNA, 22 bp, Artificial Sequence
  R/U5 forward primer
gcctcaataaagcttgccttga

SEQ ID NO: 11 - DNA, 24 bp, Artificial Sequence
  R/U5 reverse primer
tgactaaaagggtctgagggatct

SEQ ID NO: 12 - DNA, 24 bp, Artificial Sequence
  gag forward primer
ctagaacgattcgcagttaatcct

SEQ ID NO: 13 - DNA, 25 bp, Artificial Sequence
  gag reverse primer
ctatcctttgatgcacacaatagag

SEQ ID NO: 14 - DNA, 25 bp, Artificial Sequence
  egfp forward primer
caacagccacaacgtctatatcatg

SEQ ID NO: 15 - DNA, 21 bp, Artificial Sequence
  egfp reverse primer
atgttgtggcggatcttgaag

SEQ ID NO: 16 - DNA, 18 bp, Artificial Sequence
  oligomer probe specific for tRNALys3
tggcgcccgaacagggac

SEQ ID NO: 17 - protein, 626 aa, Homo sapiens
  PEPTIDE (1)..(626): SAMHD1
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQDGDVIAPLITPQKKEWNDSTSVQNPTRLREASKSRVQLFKDDPM